(19) United States
(12) Patent Application Publication
(10) Pub. No.: US 2007/0003632 A1
(43) Pub. Date: Jan. 4, 2007

(54) BURN TREATMENT FORMULATION AND METHOD OF TREATING BURNS

(76) Inventor: Jennifer Hall LAPOINTE, Epping, NH (US)

Correspondence Address: HAYES, SOLOWAY P.C., 3450 E. SUNRISE DRIVE, SUITE 140, TUCSON, AZ, 85718, US

(21) Appl. No.: 11/428,212

(22) Filed: Jun. 30, 2006

Related U.S. Application Data
(60) Provisional application No. 60/695,344, filed on Jun. 30, 2005.

(51) Int. Cl. A61K 36/898; A61K 35/64; A61K 36/47; A61K 36/736; A61K 36/886; A61K 36/28

(52) U.S. Cl. 424/539; 424/735; 424/731; 424/744; 424/764; 424/776

(57) ABSTRACT

A topical formulation for preventing radiodermatitis and/or for treatment of damaged skin comprising, in combination aloe vera gel and raw honey.

### CROSS REFERENCE TO RELATED APPLICATION

This application claims priority from U.S. Provisional Application Ser. No. 60/695,344, filed Jun. 30, 2005.

### BACKGROUND OF THE INVENTION

1. Field of the Invention

This invention relates generally to topical formulations for treatment of damaged skin. The invention has particular utility for treatment of skin damaged by the harmful effects of radiation therapy such as used in the treatment of cancer and will be described in connection with such utility, although other utilities such as topical treatment of skin damaged by exposure to the sun, as well as open sores such as bedsores, wounds, abrasions, skin ulcers and burns including chemical burns, and diaper rash.

2. Description of the Prior Art

According to the American Cancer Society close to 2.5 million new cancer cases are expected to be diagnosed in the United States this year. Based on current medical technology over half of these cancer patients will undergo some form of radiation therapy.

The primary goal of the medical community has been to prolong the life of cancer patients. Until recently, and in relation to the growing number of cancer patients that can expect to survive five years or more, the quality of life of the cancer patient has received little attention.

The onset of radiodermatitis as a result of radiation therapy in cancer patients is well documented. It is characterized by congestive or exudative redness of the skin caused by hyperthermia. Therefore, there is a need for substances effective for treating irradiated skin damage or radiodermatitis.

The topical use of anti-flammatory agents to alleviate radiodermatitis is known. Such compositions contain combinations of one or more steroidal anti-inflammatories, non-steroidal anti-inflammatories, as well as “natural” anti-inflammatories, such as an extract of aloe vera.

The bradykininase activity or ability to reduce swelling which is present in cold-processed aloe vera, with yellow sap and aloin removed, is clinically proven when used in high concentrations. See, K. Fujita, R. Teradair, and T. Nagatsu, Biochem. Pharm., Vol. 25, 205, 1976.

Several prior art patents disclose cosmetic/-therapeutic formulations including aloe vera. DeNavarre, “Non-irritating Antiperspirant” U.S. Pat. No. 4,302,443 discloses the use of aloe vera to prevent irritation.

Mihalovits, “Cosmetic Facial Preparation Containing Aloe Vera” U.S. Pat. No. 4,369,180 discloses the use of aloe vera in combination with cornstarch or cosmetic clay, albumin, allantoin, vitamin A, vitamin D2 , and vitamin E.

Millard, “Skin Treatment Preparation” U.S. Pat. No. 4,505,902 discloses a formulation which uses aloe vera juice in combination with mineral oil, apricot kernel oil, avocado oil, and cod liver oil.

Trenzeluk, “Skin Therapeutic Mixture Containing Aloe Vera Extract” U.S. Pat. No. 4,857,328 discloses a formulation using the dried leaves of the aloe vera plant as a therapeutic agent for the treatment of acne, psoriasis, burns, pimples, blackheads, and open sores.

U.S. Pat. No. 5,266,318 to Taylor-McCord teaches a formulation containing cold-processed aloe vera extract with yellow sap and aloin removed, combined with allantoin and lavender essential oil and a topical carrier for treating radiodermatitis, open sores, wounds and abrasions.

### BRIEF SUMMARY OF THE INVENTION

The present invention provides an improvement over topical solutions such as described in the above prior art. More particularly, in accordance with the present invention, there is provided a topical formulation that helps heal damaged skin while providing soothing relief. In one aspect the present invention provides a formulation for prevention of radiodermatitis and/or for treatment of radiation damaged skin comprising a combination of aloe vera gel combined with raw honey. The anti-inflammatory properties of extract of aloe vera are well documented as noted above. However, the use of raw honey for treating damaged skin has not been previously described in the art. Nor has the use of a combination of aloe vera and raw honey been previously described. Raw honey contains anti-bacterial, anti-inflammatory and anti-fungal agents. Particularly preferred is Manuka honey from New Zealand. Manuka honey is gathered in New Zealand from the Manuka bush, Leptospermum Scoparium. Another preferred honey, i.e. with similar antibacterial, anti-inflammatory and anti-fungal properties is found in Australia, and is produced from the Leptospermum Polygalifolium. However, honey from other sources also may be used.

Raw honey has antibacterial activity due primarily to hydrogen peroxide formed in a “slow release manner” by the enzyme glucose oxidase present in honey. The enzyme that produces hydrogen peroxide in honey is destroyed when honey is exposed to heat. Thus, in order to preserve the antibacterial activity, the honey should be used raw.

In order to improve hand feel and spreadability, the formulation also may include one or more plant oils. Preferred plant oils include sunflower oil, castor oil, almond oil and/or cocoa butter. Also if desired castor oil, vitamin E oil and/or rosa rubiginosa oil may be added.

Finally Cellfood gel, which is a commercially available gel comprising a mixture of aloe vera, chamomile, glycerin and a variety of trace minerals, enzymes, and amino acids, electrolytes and dissolved oxygen may be added to further enhance healing properties. If desired, a preservative such as grapefruit seed extract may be added to the mixture.

The formulation may be applied at room temperature. Preferably however, the formulation is refrigerated prior to application since the coldness further soothes the skin.

Typically, the aloe vera gel and the honey are used in a volume ratio of from 24-1 to 2-1, preferably about 12-1 to 4-1. The other ingredients, in particular the plant oils, may be used in a wide volume ratio range up to about 50-60 volume percent of the formulation.

The invention will be further described in connection with the following working examples which are given as exemplary rather than limiting:

### EXAMPLE 1

A lotion was prepared blending the following:
3 cups organic whole leaf aloe vera gel
1 oz. Cellfood gel
½ cup raw Manuka honey
40 drops rosa rubiginosa oil*
½ cup almond oil*
½ cup sunflower oil*
¼ cup castor oil*
¼ cup vitamin E oil*
   * Preferably, each of the marked ingredients are food grade, cold pressed oils.

The ingredients are blended and refrigerated until use.

### EXAMPLE 2

Mix together the following ingredients:
24 oz. organic whole leaf aloe vera gel
2 oz. sweet almond oil*
2 oz. castor oil*
6 oz. sunflower oil*
2 oz. vitamin E oil*
1 oz. Cellfood gel
2 oz. rose water
2 oz. raw Manuka honey
   * Preferably , each of the marked ingredients are food grade pressed oils.

The ingredients were blended and refrigerated until use.

### EXAMPLE 3

A cream base was made by combining two parts by volume of the lotion of Example 1 with one part by volume melted cocoa butter, one part by volume aloe vera gel and one part by volume almond oil. A small amount of rosa rubiginosa oil (10 drops) was added. The ingredients were whipped together and refrigerated prior to use.

### EXAMPLE 4

Mix together the following ingredients:
½ cup sunflower oil*
¼ cup castor oil*
¼ cup almond oil*
1 Tbs. vitamin E oil*
⅛ tsp. rosa rubiginosa oil*
2 Tbs. Cellfood gel
1 cup aloe vera gel
¼ cup raw Manuka honey

The resulting mixture was whipped and refrigerated prior to use.

### EXAMPLE 5

Mix together the following ingredients:
¾ cup sunflower oil*
¼ cup castor oil*
¼ cup almond oil*
¼ cup vitamin E oil*
2 Tbs. Cellfood gel
3 cups orgranic whole-leaf aloe vera gel
¼ cup raw Manuka honey
3 ¼ tsp. rose water
3 drops grapefruit seed extract
   * Preferably, each of the marked ingredients are food grade, cold pressed oil.

The mixture was whipped and refrigerated prior to use.

The above prepared formulations were applied topically to skin affected by radiation therapy. Application prevented radiodermatitis, provided soothing relief from burning, dryness and itching and also appeared to hasten healing of skin.

A particular advantage of the present invention is that all of the materials that make up the compositions are edible. Thus, absorption through the skin will not cause any adverse effects. This makes the formulations particularly safe to use on open sores such as bedsores, abrasions, skin ulcers and burns, and on diaper rash.

While the invention has been described in connection with preferred embodiments, various changes may be made without departing from the spirit and scope thereof.

1. A topical formulation for preventing radiodermatitis and/or for treatment of damaged skin comprising, in combination aloe vera gel and raw honey.

2. The formulation of claim 1 comprising one or more plant oils.

3. The formulation of claim 2 wherein the plant oils are selected from the group consisting of almond oil, sunflower oil, castor oil, vitamin E oil and cocoa butter.

4. The formulation of claim 1, wherein the honey comprises honey derived from Leptospermum Scoparium or Leptospermum Polygalifolium.

5. The formulation of claim 1 comprising 24-1 parts by volume of aloe vera to 2-1 parts by volume honey.

6. A method for preventing radiodermatitis which comprises applying the formulation of any of claims 1-5 to the skin prior to or following radiation therapy.

7. The method of claim 6, wherein the formulation is applied at room temperature.

8. The method of claim 6, wherein the formulation is cooled below room temperature prior to being applied to the skin.

9. The method for treating damaged skin which comprises applying the formulation of any of claims 1-5 to the damaged skin.

10. The method of claim 9, wherein the formulation is applied at room temperature.

11. The method of claim 9, wherein the formulation is cooled below room temperature prior to being applied to the skin.
